(19) United States
(12) Patent Application Publication
(10) Pub. No.: US 2019/0029582 A1
(43) Pub. Date: Jan. 31, 2019

(54) Analysis and prompting System, and Method for use with the same

(71) Applicant: CHAO-WEI CHEN, Kaohsiung City (TW)

(72) Inventor: CHAO-WEI CHEN, Kaohsiung City (TW)

(21) Appl. No.: 15/976,617

(22) Filed: May 10, 2018

(30) Foreign Application Priority Data
Jul. 31, 2017 (TW) 106125665

(51) Int. Cl. A61B 5/16; G06F 17/30; A61B 5/00; A61B 5/11

(52) U.S. Cl. CPC A61B 5/165; G06F 17/30477; A61B 5/0077; A61B 2562/0219; A61B 5/4088; A61B 5/4803; A61B 5/1113

(57) ABSTRACT

An analysis and prompting system, and method for use with the same, which, through acquisition of a behavioral situation in which a subject under analysis is situated and an actual behavior executed by the subject under analysis, and based on a set of standard situational data and a set of standard behavioral data stored in a database, analyzes whether the actual behavior of the subject under analysis executed under the behavioral situation is abnormal, and thereby determines and prompts whether the subject under analysis suffers from a cognitive disorder.

### CROSS-REFERENCE TO RELATED APPLICATIONS

This application claims the priority of Republic of China Patent Application No. 106125665 filed on Jul. 31, 2017, in the State Intellectual Property Office of the R.O.C., the disclosure of which is incorporated herein by reference.

### BACKGROUND OF THE INVENTION

### Field of the Invention

The present invention relates to technical fields of disease analysis and rehabilitative training, and more specifically to an analysis and prompting system, and method for use with the same.

### Descriptions of the Related Art

A cognitive ability refers to brain processing, storage and information extraction abilities. Nevertheless, a cognitive function disorder is a common clinical disorder, which is common in diseases such as various brain damages and dementia (such as Alzheimer's disease). As a patient suffers from a cognitive function disorder, both living quality and self-care ability will be influenced significantly to burden both family and society seriously. Moreover, a child may have issues of autistic behavior, timidity, diffidence, poor emotional control, etc. in case of poor cognitive ability.

Early symptom of cognitive disorder (e.g., senile dementia) is not apparent, so that most patients are diagnosed as suffering from intermediate and advanced illness when seeing doctors, such that treatment is delayed.

Moreover, for cognitive disorder disease, there is a lack of effective treatment method at present, so that need of nursing resources for taking care of and treating patients becomes huge burden for families and the society.

In light of above, how to provide a technology which may assist in determining whether an action person has a cognitive disorder, and assist patients in performing rehabilitative training is the technical subject of the present invention to be solved.

### SUMMARY OF THE INVENTION

In view of the above drawbacks in the conventional technology, a primary object of the present invention is to provide an analysis and prompting system, and method for use with the same, which may analyze and prompt whether an actual behavior of a subject under analysis is abnormal.

Another object of the present invention is to provide an analysis and prompting system, and method for use with the same, which have a function of prompting a standard behavior, to assist a patient with cognitive disorder in performing rehabilitative training.

To achieve the above and other objects, an analysis and prompting system is provided in the present invention, which analyzes and prompts whether an actual behavior executed by a subject under analysis under a behavioral situation is abnormal, including: a database, which stores a set of standard situational data and a set of standard behavioral data in correspondence, the standard situational data representing the behavioral situation, the standard behavioral data representing a standard behavior which has to be executed under the behavioral situation; an acquisition module, which acquires the behavioral situation in which the subject under analysis is situated, and generates a set of actual situational data according thereto, as well as acquires the actual behavior executed by the subject under analysis, and generates a set of actual behavioral data according thereto; an analysis module, which searches for standard situational data which match with the actual situational data from the database, and retrieves standard behavioral data in correspondence with the standard situational data, for determining whether the actual behavior executed by the subject under analysis matches with the standard behavior which has to be executed under the behavioral situation by comparing the standard behavioral data and the actual behavioral data, in order to analyze whether the actual behavior executed by the subject under analysis is abnormal; and a prompt module, which prompts an analysis result of the analysis module.

Preferably, the analysis and prompting system said above, wherein, may further include a guiding module, which causes the prompt module to prompt the standard behavior which has to be executed under the behavioral situation in accordance with the standard behavioral data, to guide match of the actual behavior executed by the subject under analysis with the standard behavior executed under the behavioral situation.

Preferably, the analysis and prompting system said above, wherein, the standard behavior may comprise multiple consecutive standard actions, while the actual behavior may comprise multiple consecutive actual actions; the guiding module guides the multiple consecutive actual actions of the actual behavior to match with the multiple consecutive standard actions of the standard behavior.

Preferably, the analysis and prompting system said above, wherein, may further include a consultation module, which, when the actual behavior executed by the subject under analysis is abnormal, causes the prompt module to prompt the standard behavior which has to be executed under the behavioral situation in accordance with the standard behavioral data, to guide match of the actual behavior executed by the subject under analysis with the standard behavior executed under the behavioral situation.

Preferably, the analysis and prompting system said above, wherein, the standard behavior may comprise multiple consecutive standard actions, while the actual behavior may comprise multiple consecutive actual actions; the consultation module guides the multiple consecutive actual actions of the actual behavior to match with the multiple consecutive standard actions of the standard behavior.

Preferably, the analysis and prompting system said above, wherein, the acquisition module may comprise at least one of an optical sensor, a locating sensor, a voice sensor and a gravity sensor.

Preferably, the analysis and prompting system said above, wherein, the behavioral situation may refer to an interaction situation between the subject under analysis and an interaction subject; the actual behavior may refer to the actual behavior executed by the subject under analysis for the interaction subject; the standard behavior may refer to the standard behavior executed by the subject under analysis for the interaction subject.

Preferably, the analysis and prompting system said above, wherein, the acquisition module may acquire and use the actual behavior executed by the interaction subject as the actual situational data for the analysis module to define the behavioral situation in which the subject under analysis is situated according thereto.

Preferably, the analysis and prompting system said above, wherein, when the analysis module analyzes that the actual behavior executed by the subject under analysis is abnormal, the database may be caused to store the actual situational data and the actual behavioral data for the prompt module to prompt the analysis result of the analysis module subsequently.

Preferably, the analysis and prompting system said above, wherein, may further include a learning module, which, when the actual behavior executed by the subject under analysis is normal, may store the corresponding actual situational data and actual behavioral data in the database as the corresponding standard situational data and standard behavioral data stored in the database.

Preferably, the analysis and prompting system said above, wherein, the subject under analysis may be a cognitive disorder patient.

Preferably, the analysis and prompting system said above, wherein, the prompt module may prompt the analysis result of the analysis module through at least one of a voice medium and an image medium.

The present invention further provides an analysis and prompting method, which analyzes and prompts whether an actual behavior executed by a subject under analysis under a behavioral situation is abnormal, including: providing a database for storing a set of standard situational data and a set of standard behavioral data in correspondence, the standard situational data representing the behavioral situation, the standard behavioral data representing a standard behavior which has to be executed under the behavioral situation; providing an acquisition module to acquire the behavioral situation in which the subject under analysis is situated, and generate a set of actual situational data according thereto, as well as acquire the actual behavior executed by the subject under analysis, and generate a set of actual behavioral data according thereto; and providing an analysis module to search for standard situational data which match with the actual situational data from the database, and retrieve standard behavioral data in correspondence with the standard situational data from the database, for determining whether the actual behavior executed by the subject under analysis matches with the standard behavior which has to be executed under the behavioral situation by comparing the standard behavioral data and the actual behavioral data, in order to analyze whether the actual behavior executed by the subject under analysis is abnormal, and prompt the analysis result of the analysis module.

Preferably, the analysis and prompting method said above, wherein, may further include prompting the standard behavior which has to be executed under the behavioral situation in accordance with the standard behavioral data, to guide match of the actual behavior executed by the subject under analysis with the standard behavior executed under the behavioral situation.

Preferably, the analysis and prompting method said above, wherein, may further include prompting the standard behavior which has to be executed under the behavioral situation in accordance with the standard behavioral data, to guide match of the actual behavior executed by the subject under analysis with the standard behavior executed under the behavioral situation.

Preferably, the analysis and prompting method said above, wherein, may further include prompting the standard behavior which has to be executed under the behavioral situation in accordance with the standard behavioral data when the actual behavior executed by the subject under analysis is abnormal, to guide match of the actual behavior executed by the subject under analysis with the standard behavior executed under the behavioral situation.

Preferably, the analysis and prompting method said above, wherein, the behavioral situation may refer to an interaction situation between the subject under analysis and an interaction subject; the actual behavior may refer to the actual behavior executed by the subject under analysis for the interaction subject; the standard behavior may refer to the standard behavior executed by the subject under analysis for the interaction subject.

Preferably, the analysis and prompting method said above, wherein, may further include providing the acquisition module to acquire and use the actual behavior executed by the interaction subject to define the behavioral situation in which the subject under analysis is situated.

Preferably, the analysis and prompting method said above, wherein, when the analysis module analyzes that the actual behavior executed by the subject under analysis is abnormal, the database may be caused to store the actual situational data and the actual behavioral data for the prompt module to prompt the analysis result of the analysis module subsequently.

Preferably, the analysis and prompting method said above, wherein, when the actual behavior executed by the subject under analysis is normal, stores the corresponding actual situational data and actual behavioral data in the database as the corresponding standard situational data and standard behavioral data stored in the database.

Preferably, the analysis and prompting method said above, wherein, the analysis module searches for the standard situational data which match with the actual situational data from the database to predict a behavior which is going to be executed by the subject under analysis.

In comparison to prior arts, an analysis and prompting system, and method for use with the same is provided according to the present invention, which acquires a behavioral situation in which a subject under analysis is situated, and an actual behavior executed by the subject under analysis under the behavioral situation, to search out a standard situational data which match with the behavioral situation from a database, as well as identifies whether standard behavioral data corresponding to the standard situational data stored in the database match with the acquired actual behavior of the subject under analysis through comparison to analyze whether the actual behavior is abnormal. Thereby, the present invention may assist in determining whether there is cognitive disorder for the subject under analysis, so that an illness of the subject under analysis is discovered as soon as possible, in order to avoid delayed treatment.

Moreover, the present invention may further provide a function which prompts a standard behavior for the subject under analysis by identifying a behavioral situation in which the subject under analysis is situated presently, in order to assist a cognitive disorder patient in performing a rehabilitative training for the cognitive disorder patient to be capable of basic self-living.

### BRIEF DESCRIPTION OF THE DRAWINGS

The above and other aspects, features and other advantages of the present invention will be more clearly understood from the following detailed description taken in conjunction with the accompanying drawings, in which:

FIG. 1 is an architecture diagram showing a first embodiment of an analysis and prompting system according to the present invention.

FIG. 2 is an architecture diagram showing a second embodiment of an analysis and prompting system according to the present invention.

FIG. 3 is an architecture diagram showing a third embodiment of an analysis and prompting system according to the present invention.

FIG. 4 is an architecture diagram showing a fourth embodiment of an analysis and prompting system according to the present invention.

FIG. 5 is a basic flowchart showing an analysis and prompting method according to the present invention.

### DETAILED DESCRIPTION OF THE PREFERRED EMBODIMENT

Embodiments of the present invention will now be described in detail with reference to the accompanying drawings. The present invention may, however, be embodied in many different forms and should not be construed as being limited to the embodiments set forth herein. Rather, these embodiments are provided so that this disclosure will be thorough and complete, and will fully convey the scope of the present invention to those skilled in the art. In the drawings, the shapes and dimensions of elements may be exaggerated for clarity, and the same reference numerals will be used throughout to designate the same or like components.

An analysis and prompting system according to the present invention is used to analyze and prompt, under a behavioral situation, whether an actual behavior executed by a subject under analysis under the behavioral situation is abnormal. It should be noted that the subject under analysis is a natural person, particularly a cognitive disorder patient. However, the subject under analysis may also be electrical equipment.

As shown in FIG. 1, an analysis and prompting system 1 includes a database 11, an acquisition module 12, an analysis module 13 and a prompt module 14.

The database 11 is used to store corresponding standard situational data and standard behavioral data, wherein the standard situational data may represent a behavioral situation, while the standard behavioral data may represent a standard behavior which has to be executed under the behavioral situation, the standard behavioral data may include data acquired by the acquisition module 12 from a normal action person, and data after automatic or manual analysis performed for the acquired data. Wherein, the standard behavior may be multiple consecutive standard actions, or a language in compliance with standard syntax or logical relationship.

In addition, the database 11 may store file data in other formats as need. For example, the data stored in the database 11 may be data in format of image which represents standard actions. The data in format of image may support at least the formats of AVI, MOV, MPEG/MPG/DAT, RM and WMV. The database 11 may also store data in format of audio for representing language. The data in format of audio may support at least the formats of MP3, WMA, WAV, ASF, AAC, APE and FLAC.

More specifically, the behavioral situation refers to an interaction situation between the subject under analysis and an interaction subject, the actual behavior refers to the actual behavior executed by the subject under analysis for the interaction subject, and the standard behavior refers to the standard behavior executed by the subject under analysis for the interaction subject. Wherein, the interaction subject may be a natural person, the behavioral situation may be a linguistic interaction between a cognitive disorder patient and others. However, the interaction subject may also be an object subject to interact with a cognitive disorder patient. The object is either, for example, an electrical equipment for daily housekeeping, such as laundry machine, air conditioner, microwave oven etc., or a commercial electrical equipment, such as vending machine, self-service laundry machine etc.

The acquisition module 12 is used to acquire the behavioral situation in which the subject under analysis is situated, and generate the actual situational data according thereto, as well as acquire the actual behavior executed by the subject under analysis, and generate the actual behavioral data according thereto.

In one embodiment of the present invention, the acquisition module 12 is a sensor module which may acquire daily life information of the subject under analysis. The sensor module may be composed of an optical sensor, a locating sensor, a voice sensor, a gravity sensor, or a combination of at least two sensors mentioned above. Furthermore, the acquisition module 12 may be a location fixed sensor which is fixed in a field space (e.g., a room in a house, or an office), or a portable sensor worn on the subject under analysis.

The acquisition module 12 may comprise multiple cameras or multiple audio detectors arranged in different rooms separately for acquiring activity behaviors of the subject under analysis in different rooms, and handling habits of the subject under analysis in daily life by integrating activity behavior information acquired by the acquisition module 12 in different rooms. However, the acquisition module 12 may also comprise electronic sensors installed on different electrical equipment (e.g., household appliances, such as laundry machine, air conditioner, electric cooker, microwave oven etc.) for acquiring action steps of the subject under analysis who operates the electrical equipment.

In addition, the acquisition module 12 may further comprise a wearable camera capable, an audio detector, a spatial locator or a stereoscopic gyroscope etc., which have wireless transmission function. Such that the present invention may not only be used at home to acquire home life information of the subject under analysis, but also be used in public to acquire social life information of the subject under analysis. Moreover, the acquisition module 12 may further comprise a wearable physiologic signal monitor for acquiring physiologic signals of the subject under analysis, such as heartbeat or blood pressure etc.

Therefore, the acquisition module 12 may perform an arbitrary combination of members with different functions based on different requirements of use. By way of examples: (1) indoor coordinates and location change of the subject under analysis may be acquired through a combination of an indoor camera, a wearable camera and a wearable spatial locator; (2) actions due to activities of limbs of the subject under analysis may be acquired through a combination of an indoor camera, a wearable camera and a wearable stereoscopic gyroscope; (3) an interaction between the subject under analysis and an electrical equipment may be acquired through a combination of an indoor camera, a wearable camera, and an electronic sensor installed on the electrical equipment; (4) a conversation and an interaction between the subject under analysis and other individuals may be acquired through a combination of an indoor camera, a wearable camera, an indoor audio detector, a wearable audio detector, a wearable spatial locator and a wearable stereoscopic gyroscope.

The actual behavioral data, which are generated by the acquisition module 12, are subject to automatic analysis and/or manual aided analysis, and then may be integrated to be data for representing various kinds of pattern information (behavioral pattern data) of the subject under analysis, including, but not limited to, the following types of data: (1) data representing action execution function, such as recording how the subject under analysis executes specific targeted action steps (how to call, how to boil water etc.); (2) data representing linguistic function, such as recording features of conversation and textual communication and features of used vocabularies and grammars when the subject under analysis desires to express a specific goal; (3) data representing spatial locating function, such as recording paths when the subject under analysis desires to reach a specific place (e.g., supermarket, food market, bank); (4) data representing facial recognition function, such as recording interactions between the subject under analysis and other people in order to create human relationship data in correspondence with the subject under analysis.

The analysis module 13 searches for the standard situational data which match with the actual situational data acquired by the acquisition module 12 from the database 11 to predict a behavior which is going to be executed by the subject under analysis, and retrieves the standard behavioral data in correspondence with the standard situational data in the database 11, for determining whether the actual behavior executed by the subject under analysis matches with the standard behavior which has to be executed under the behavioral situation by comparing the retrieved standard behavioral data and the actual behavioral data acquired by the acquisition module 12, so as to analyze whether the actual behavior executed by the subject under analysis is abnormal.

The prompt module 14 prompts the analysis result of the analysis module 13 through a voice medium and/or an image medium. That is, the prompt module 14 may output the analysis result in a manner of voice prompt, or display the analysis result in a manner of image output.

Specifically, the acquisition module 12 may further acquire and use the actual behavior executed by the interaction subject as the actual situational data for the analysis module 13 to define the behavioral situation in which the subject under analysis is situated according thereto. When the subject under analysis interacts with an electrical equipment, the acquisition module 12 may acquire the actual behavior executed by the electrical equipment, such as in a manner of capture done by a camera, or through an operation related to sensing the electrical equipment by an electronic sensor on the electrical equipment, and may use the actual behavior executed by the electrical equipment as the actual situational data for the analysis module 13 to define the behavioral situation (i.e., the interaction situation between the subject under analysis and the electrical equipment) in which the subject under analysis is situated presently by identifying the interaction subject (i.e., electrical equipment of laundry machine, for example).

Furthermore, the present invention may also accomplish the analysis of behavioral situation for the subject under analysis in combination with an image recognition technology. For example, the acquisition module 12 is a camera used to acquire image data of the interaction subject for the analysis module 13 to identify that an electrical equipment is included in the image data generated by the acquisition module 12 through the image recognition technology, in order for defining that the subject under analysis is in the interaction situation with the electrical equipment at present according thereto.

Moreover, when the analysis module 13 analyzes that the actual behavior executed by the subject under analysis is abnormal, that is, the actual behavioral data of the subject under analysis mismatch with the standard behavioral data stored in the database 11, the database 11 is caused to store the actual situational data and the actual behavioral data in which the aforementioned determination result is abnormal for the prompt module 14 to prompt the analysis result of the analysis module 13 subsequently, in order for a doctor to browse the actual behavioral data, which show that the subject under analysis is abnormal, output by the prompt module 14 as a reference for related disease diagnosis.

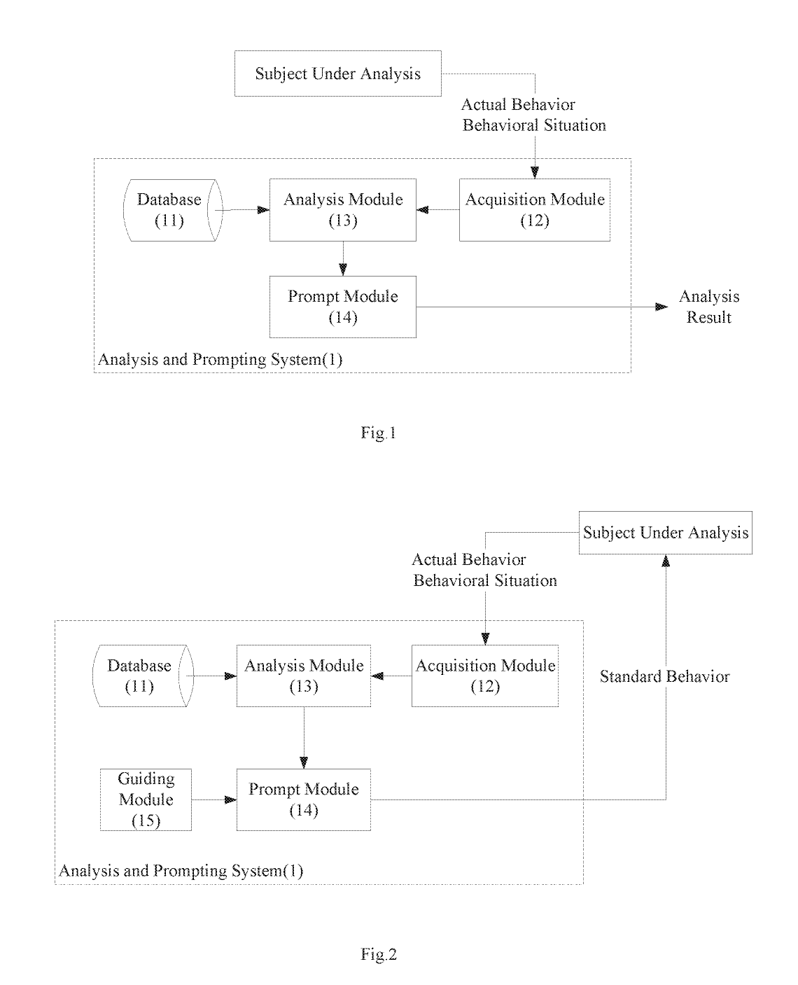

Refer to FIG. 2 in conjunction. The analysis and prompting system 1 may further include a guiding module 15 for causing the prompt module 14 to prompt the standard behavior which has to be executed under the behavioral situation in accordance with the standard behavioral data, to guide match of the actual behavior executed by the subject under analysis with the standard behavior executed under the behavioral situation. Wherein, the standard behavior stored in the database 11 comprises multiple consecutive standard actions, and the actual behavior acquired by the acquisition module also comprises multiple consecutive actual actions. The guiding module 15 is used for causing the prompt module 14 to prompt the standard behavioral data according to the standard situational data, which match with the actual situational data, searched out by the analysis module 13, and the standard behavioral data corresponding to the standard situational data, retrieved by the analysis module 13, so as to guide the subject under analysis for executing multiple consecutive actual actions to match with the multiple consecutive standard actions of the standard behavior, in order to aid the subject under analysis in performing rehabilitative training for self-living.

For example, when the analysis module 13 analyzes that a cognitive disorder patient (i.e., the subject under analysis) enters a kitchen at the time close to dinner time according to acquisition data of the acquisition module 12, or when the analysis module 13 identifies an interaction subject of an “electric pot” according to image data acquired by the acquisition module 12, the analysis module 13 analyzes that the cognitive disorder patient desires to perform a “cooking” activity at present. In the meantime, the analysis module 13 may retrieve operation steps related to “cooking” from the database 11, such that the prompt module 14 may prompt how to perform “cooking” (including cooking steps of rice cleaning, placement of rice in an electric cooker, activation of the electric cooker etc.) in a manner of image or voice for the cognitive disorder patient.

Further, for example, when the analysis module 13 analyzes that the cognitive disorder patient talks with others, vocabularies and grammars which match with conversation object and purpose may be recommended to the cognitive disorder patient according to conversation content of both parties acquired by the acquisition module 12, and linguistic data stored in the database 11. (For example, when an expression of the cognitive disorder patient to a daughter with respect to pain in knees is acquired, correct vocabularies and grammars for expression of uncomfortable knees/lower limbs are prompted.)

Refer to FIG. 3 in conjunction. The analysis and prompting system 1 may further include a consultation module 16, which is used for, when the analysis module 13 analyzes that the actual behavior executed by the subject under analysis is abnormal, causing the prompt module 14 to prompt the standard behavior which has to be executed under the behavioral situation in accordance with the standard behavioral data, to guide match of the actual behavior executed by the subject under analysis with the standard behavior executed under the behavioral situation. Wherein, the standard behavior stored in the database 11 comprises multiple consecutive standard actions, and the actual behavior acquired by the acquisition module 12 also comprises multiple consecutive actual actions. The consultation module 16 is used for guiding the subject under analysis for executing multiple consecutive actual actions of the actual behavior to match with the multiple consecutive standard actions of the standard behavior.

In other words, the basic function of the consultation module 16 is similar to that of the guiding module 15 show in FIG. 2 with one of the exception in that the guiding module 15 will cause the prompt module 14 to prompt the standard behavioral data retrieved by the analysis module 13 despite whether the actual behavior of the subject under analysis is abnormal, while the consultation module 16 will cause the prompt module 14 to prompt the standard behavioral data retrieved by the analysis module 13 only when the analysis module 13 analyzes that the actual behavior executed by the subject under analysis at present is abnormal, that is, analyzes that incorrect operation steps occur in the executed multiple consecutive actual actions, in order for reminding the subject under analysis to correct the incorrect steps which occur (for example, the cognitive disorder patient is reminded to adjust water amount when the water amount added in the electric cooker differs from a water amount in a standard approach).

In contrast, a standard behavior guiding mode provided by the guiding module 15 illustrated in FIG. 2 is applicable relatively to a cognitive disorder patient with a severer symptom, while a standard behavior consultation mode provided by the consultation module 16 illustrated in FIG. 3 is applicable relatively to a cognitive disorder patient with a lighter symptom. Thus, the analysis and prompting system 1 according to the present invention is applicable to different symptom of cognitive disorder patients for use.

However, the guiding module 15 and the consultation module 16 may also be used in combination. For example, when the subject under analysis is in a preparation action prior to execution of consecutive actions, the analysis module 13 of the analysis and prompting system 1 searches for the standard situational data which match with the actual situational data from the database 11 according to the actual situational data acquired by the acquisition module 12, and determines subsequent consecutive actions which might be executed by the subject under analysis according to the standard behavioral data, which comprise multiple consecutive standard actions, corresponding to the standard situational data stored in the database 11. In the meantime, the guiding module 15 may query whether the multiple consecutive standard actions included in the standard behavioral data have to be prompted, and offer a prompt, or wait for the subject under analysis to execute subsequent actions.

When the subject under analysis starts executing the consecutive actions, the analysis module 13 of the analysis and prompting system 1 determines that the subject under analysis starts executing the consecutive actions by the actual behavioral data generated by the acquisition module 12, followed by causing the guiding module 15 to prompt the multiple consecutive standard actions in the standard behavioral data by the prompt module 14 in order. When an incorrect order occurs for the consecutive actions of the subject under analysis, the analysis module 13 of the analysis and prompting system 1 determines that the incorrect order occurs for the consecutive actions executed by the subject under analysis by means of the actual behavioral data generated by the acquisition module 12, followed by causing the consultation module 16 to prompt how to correct for recovering the correct order of behavior by the prompt module 14.

For example, when the subject under analysis stands in front of a wash basin, the analysis module 13 of the analysis and prompting system 1 searches for the standard situational data which match with the actual situational data from the database 11 according to the actual situational data acquired by the acquisition module 12, to determine that the subject under analysis might execute (1) tooth brushing or (2) face washing subsequently, and extract the standard behavioral data corresponding to tooth brushing or face washing from the database 11. After that, the guiding module 15 is caused to query whether there is a need to prompt an order of behavior for tooth brushing or face washing by the prompt module 14. When the subject under analysis takes out a toothbrush to start tooth brushing, the analysis module 13 of the analysis and prompting system 1 determines that the subject under analysis starts tooth brushing by the actual behavioral data generated by the acquisition module 12, and causes the guiding module 15 to prompt an order of behavior for tooth brushing by the prompt module 14. When an incorrect order occurs with respect to tooth brushing for the subject under analysis, such as starting tooth brushing without squeezing toothpaste, the analysis module 13 of the analysis and prompting system 1 determines that the incorrect order occurs with respect to tooth brushing for the subject under analysis by means of the actual behavioral data generated by the acquisition module 12, followed by causing the consultation module 16 to prompt correct behavioral data of tooth brushing by the prompt module 14.

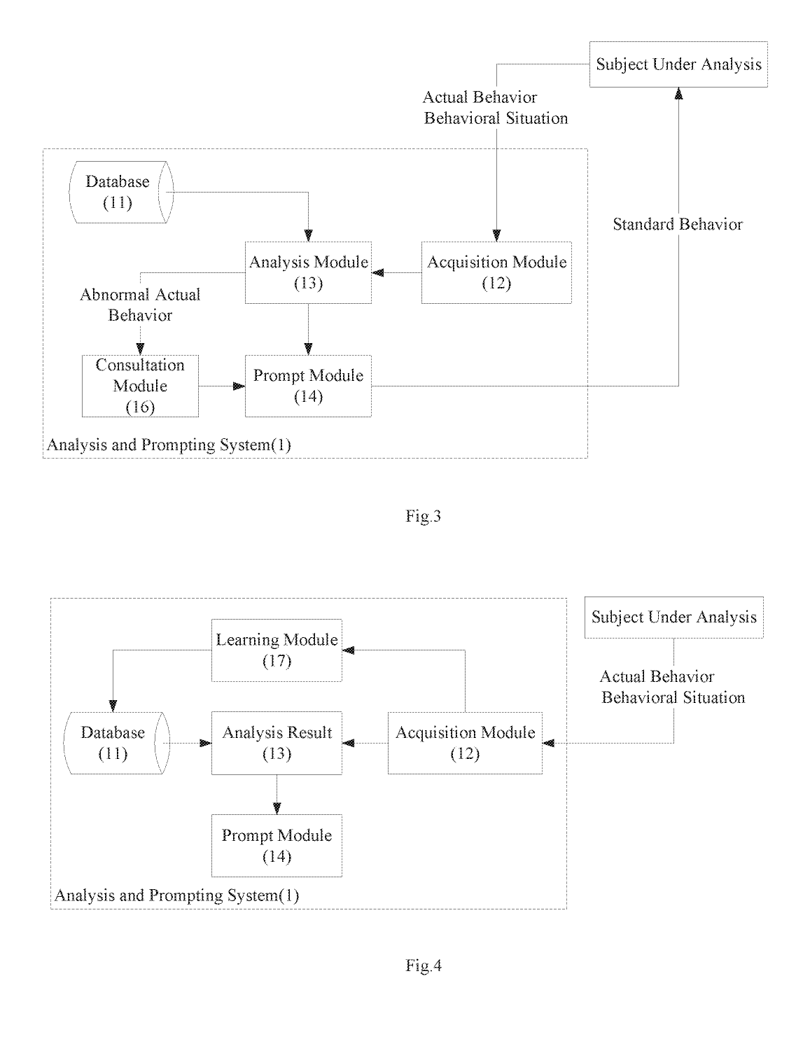

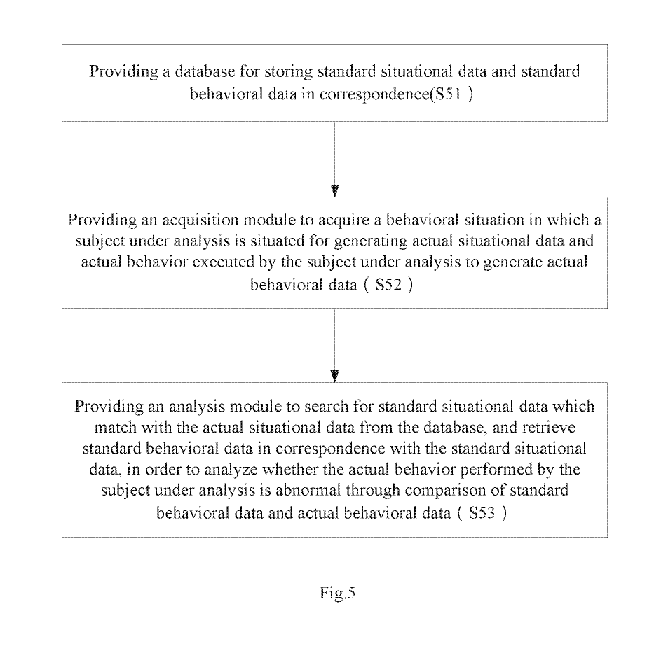

Referring to FIG. 4, the analysis and prompting system 1 may further include a learning module 17, wherein, when the actual behavior executed by the subject under analysis is normal, the learning module 17 is used to store the corresponding actual situational data and actual behavioral data generated by the acquisition module 12 in the database 11 as the corresponding standard situational data and standard behavioral data stored in the database 11. That is, collection of the standard situational data and standard behavioral data by the learning module 17 includes collection of life information of a normal action person, though, for example, recording of operations required to be executed when cooking and an order thereof, acquisition of activity information for limbs of the normal action person, and information of her/his interaction with an electric pot, as well as performing integration to generate pattern information for completed “cooking” execution function (including upfront steps of cooking, i.e., the process steps for predicting when to start cooking and to cook).

Refer to FIG. 5 in conjunction. The analysis and prompting method according to the present invention is used to analyze and prompt whether an actual behavior executed by a subject under analysis is abnormal, including the following flow of process steps:

At first, step S51 is executed: A database is provided for storing standard situational data and standard behavioral data in correspondence, wherein the standard situational data may represent a behavioral situation, the standard behavioral data may represent a standard behavior which has to be executed under the behavioral situation, followed by executing step S52.

In one embodiment, the standard behavior may be multiple consecutive standard actions, or a language in compliance with standard syntax or logical relationship. Furthermore, the database may store file data in other formats as need. Correspondingly, the data stored in the database may be data in format of image which represents standard actions, or the database may also store data in format of audio for representing language.

More specifically, the behavioral situation refers to an interaction situation between the subject under analysis and an interaction subject, the actual behavior refers to the actual behavior executed by the subject under analysis for the interaction subject, and the standard behavior refers to the standard behavior executed by the subject under analysis for the interaction subject. Wherein, the interaction subject may be a natural person, the behavioral situation may be a linguistic interaction between a cognitive disorder patient and others. However, the interaction subject may also be an object. The object is either, for example, an electrical equipment for daily housekeeping, such as laundry machine, air conditioner, microwave oven etc., or a commercial electrical equipment, such as vending machine, self-service laundry machine etc.

In step S52: An acquisition module is provided to acquire the behavioral situation in which the subject under analysis is situated, and generate the actual situational data according thereto, as well as acquire the actual behavior executed by the subject under analysis, and generate the actual behavioral data according thereto. Then, step S53 is executed.

More specifically, the acquisition module is a sensor module which may acquire daily life information of the subject under analysis. The sensor module may be composed of an optical sensor, a locating sensor, a voice sensor, a gravity sensor, or a combination of at least two sensors mentioned above. Furthermore, the acquisition module 12 may be a location fixed sensor which is fixed in a field space (e.g., a room in a house, or an office), or a portable sensor worn on the subject under analysis.

In step S53: An analysis module is provided to search for standard situational data which match with the actual situational data from the database, and retrieve standard behavioral data in correspondence with the standard situational data, for determining whether the actual behavior executed by the subject under analysis matches with the standard behavior which has to be executed under the behavioral situation by comparing the standard behavioral data and the actual behavioral data, in order to analyze whether the actual behavior executed by the subject under analysis is abnormal, and prompt the analysis result of the analysis module.

In one embodiment, the analysis and prompting method further includes providing the acquisition module to acquire and use the actual behavior executed by the interaction subject to define the behavioral situation in which the subject under analysis is situated, and then retrieves standard behavioral data in correspondence with the standard situational from the database, in order to analyze whether the actual behavior executed by the subject under analysis is abnormal.

Furthermore, the acquisition module may further acquire and use the actual behavior executed by the interaction subject as the actual situational data for the analysis module to define the behavioral situation in which the subject under analysis is situated according thereto. When the subject under analysis interacts with an electrical equipment, the acquisition module may acquire the actual behavior executed by the electrical equipment through acquisition of the actual behavior executed by the electrical equipment, such as in a manner of capture done by a camera, or through an operation related to sensing the electrical equipment by an electronic sensor on the electrical equipment, and may use the actual behavior as the actual situational data for the analysis module to define the behavioral situation in which the subject under analysis is situated presently (i.e., the interaction situation between the subject under analysis and the electrical equipment) by identifying the interaction subject (i.e., electrical equipment of laundry machine, for example).

Furthermore, the present invention may also accomplish the analysis of behavioral situation for the subject under analysis in combination with an image recognition technology. For example, the acquisition module is a camera used to acquire image data of the interaction subject for the analysis module to identify that a wash machine is included in the image data generated by the acquisition module through the image recognition technology, in order for defining that the subject under analysis is in the interaction situation with the wash machine at present according thereto, and then retrieves standard behavioral data of a “laundry machine” in correspondence with the standard situational data from the database, in order to analyze whether the actual laundry behavior executed by the subject under analysis is abnormal.

In one embodiment, the analysis and prompting method may further include prompting the standard behavior which has to be executed under the behavioral situation in accordance with the standard behavioral data, to guide match of the actual behavior executed by the subject under analysis with the standard behavior executed under the behavioral situation.

For example, when the analysis module analyzes that a cognitive disorder patient (i.e., the subject under analysis) enters a kitchen at the time close to dinner time according to acquisition data of the acquisition module, or when the analysis module identifies an interaction subject of an “electric pot” according to image data acquired by the acquisition module, it analyzes that the cognitive disorder patient desires to perform a “cooking” activity at present. In the meantime, the analysis module may retrieve operation steps related to “cooking” from the database, such that the prompt module may prompt how to perform “cooking” (including cooking steps of rice cleaning, placement of rice in an electric cooker, activation of the electric cooker etc.) in a manner of image or voice for the cognitive disorder patient.

In one embodiment, the analysis and prompting method may further include prompting the standard behavior which has to be executed under the behavioral situation in accordance with the standard behavioral data when the actual behavior executed by the subject under analysis is abnormal, to guide match of the actual behavior executed by the subject under analysis with the standard behavior executed under the behavioral situation. In other words, only when the analysis module analyzes that the actual behavior executed by the subject under analysis at present is abnormal, that is, analyzes that incorrect operation steps occur in the executed multiple consecutive actual actions, according to the standard behavioral data to remind the subject under analysis to correct the incorrect steps which occur (for example, the cognitive disorder patient is reminded to adjust water amount when the water amount added in the electric cooker differs from a water amount in a standard approach).

In one embodiment, when the analysis module analyzes that the actual behavior executed by the subject under analysis is abnormal, the database is caused to store the actual situational data and the actual behavioral data for the prompt module to prompt the analysis result of the analysis module subsequently. As a result, the physician can use the actual behavioral data of the subject under analysis by browsing the data as a reference for the diagnosis of the related diseases.

In one embodiment, when the actual behavior executed by the subject under analysis is normal, stores the corresponding actual situational data and actual behavioral data in the database as the corresponding standard situational data and standard behavioral data stored in the database. In this way, standard situation data and standard behavioral data are collected to provide follow-up analysis and judgment basis for whether the actual behavior is abnormal or not.

In conclusion, a system, and method for use with the same is provided according to the present invention, which acquires a behavioral situation in which a subject under analysis is situated, and an actual behavior executed by the subject under analysis under the behavioral situation, to search out a standard situational data which match with the behavioral situation from a database, as well as identifies whether standard behavioral data corresponding to the standard situational data stored in the database match with the acquired actual behavior of the subject under analysis are abnormal through comparison. Thereby, the present invention may assist in determining whether there is cognitive disorder for the subject under analysis, so that an illness of the subject under analysis is discovered as soon as possible, in order to avoid delayed treatment.

Moreover, the present invention may further provide a function which prompts a standard behavior for the subject under analysis by identifying a behavioral situation in which the subject under analysis is situated presently, in order to assist a cognitive disorder patient in performing a rehabilitative training for the cognitive disorder patient to be capable of basic self-living. The function which prompts the standard behavior mentioned above may be divided into a consultation mode and a guiding mode according to illness severity of the cognitive disorder patient, to meet requirements of use for different levels of cognitive disorder patients.

The examples above are only illustrative to explain principles and effects of the present invention, but not to limit the present invention. It will be apparent to those skilled in the art that modifications and variations can be made without departing from the spirit and scope of the present invention. Therefore, the protection range of the rights of the present invention should be as defined by the appended claims.

What is claimed is:

1. An analysis and prompting system, which analyzes and prompts whether an actual behavior executed by a subject under analysis under a behavioral situation is abnormal, including:
a database, which stores a set of standard situational data and a set of standard behavioral data in correspondence, the standard situational data representing the behavioral situation, the standard behavioral data representing a standard behavior which has to be executed under the behavioral situation;
an acquisition module, which acquires the behavioral situation in which the subject under analysis is situated, and generates a set of actual situational data according thereto, as well as acquires the actual behavior executed by the subject under analysis, and generates a set of actual behavioral data according thereto;
an analysis module, which searches for standard situational data which match with the actual situational data from the database, and retrieves standard behavioral data in correspondence with the standard situational data from the database, for determining whether the actual behavior executed by the subject under analysis matches with the standard behavior which has to be executed under the behavioral situation by comparing the standard behavioral data and the actual behavioral data, in order to analyze whether the actual behavior executed by the subject under analysis is abnormal; and
a prompt module, which prompts an analysis result of the analysis module.

2. The analysis and prompting system according to claim 1, further including a guiding module, which causes the prompt module to prompt the standard behavior which has to be executed under the behavioral situation in accordance with the standard behavioral data, to guide match of the actual behavior executed by the subject under analysis with the standard behavior executed under the behavioral situation.

3. The analysis and prompting system according to claim 2, wherein the standard behavior comprises multiple consecutive standard actions, while the actual behavior comprises multiple consecutive actual actions; the guiding module guides the multiple consecutive actual actions of the actual behavior to match with the multiple consecutive standard actions of the standard behavior.

4. The analysis and prompting system according to claim 1, further including a consultation module, which, when the actual behavior executed by the subject under analysis is abnormal, causes the prompt module to prompt the standard behavior which has to be executed under the behavioral situation in accordance with the standard behavioral data, to guide match of the actual behavior executed by the subject under analysis with the standard behavior executed under the behavioral situation.

5. The analysis and prompting system according to claim 4, wherein the standard behavior comprises multiple consecutive standard actions, while the actual behavior comprises multiple consecutive actual actions; the consultation module guides the multiple consecutive actual actions of the actual behavior to match with the multiple consecutive standard actions of the standard behavior.

6. The analysis and prompting system according to claim 1, wherein the acquisition module comprises at least one of an optical sensor, a locating sensor, a voice sensor and a gravity sensor.

7. The analysis and prompting system according to claim 1, wherein the behavioral situation refers to an interaction situation between the subject under analysis and an interaction subject; the actual behavior refers to the actual behavior executed by the subject under analysis for the interaction subject; the standard behavior refers to the standard behavior executed by the subject under analysis for the interaction subject.

8. The analysis and prompting system according to claim 7, wherein the acquisition module acquires and uses the actual behavior executed by the interaction subject as the actual situational data for the analysis module to define the behavioral situation in which the subject under analysis is situated according thereto.

9. The analysis and prompting system according to claim 1, wherein, when the analysis module analyzes that the actual behavior executed by the subject under analysis is abnormal, the database is caused to store the actual situational data and the actual behavioral data for the prompt module to prompt the analysis result of the analysis module subsequently.

10. The analysis and prompting system according to claim 1, further including a learning module, which, when the actual behavior executed by the subject under analysis is normal, stores the corresponding actual situational data and actual behavioral data in the database as the corresponding standard situational data and standard behavioral data stored in the database.

11. The analysis and prompting system according to claim 1, wherein the subject under analysis is a cognitive disorder patient.

12. The analysis and prompting system according to claim 1, wherein the prompt module prompts the analysis result of the analysis module through at least one of a voice medium and an image medium.

13. An analysis and prompting method, which analyzes and prompts whether an actual behavior executed by a subject under analysis under a behavioral situation is abnormal, including:
providing a database for storing a set of standard situational data and a set of standard behavioral data in correspondence, the standard situational data representing the behavioral situation, the standard behavioral data representing a standard behavior which has to be executed under the behavioral situation;
providing an acquisition module to acquire the behavioral situation in which the subject under analysis is situated, and generate a set of actual situational data according thereto, as well as acquire the actual behavior executed by the subject under analysis, and generate a set of actual behavioral data according thereto; and
providing an analysis module to search for standard situational data which match with the actual situational data from the database, and retrieve standard behavioral data in correspondence with the standard situational data from the database, for determining whether the actual behavior executed by the subject under analysis matches with the standard behavior which has to be executed under the behavioral situation by comparing the standard behavioral data and the actual behavioral data, in order to analyze whether the actual behavior executed by the subject under analysis is abnormal, and prompt the analysis result of the analysis module.

14. The analysis and prompting method according to claim 13, further including prompting the standard behavior which has to be executed under the behavioral situation in accordance with the standard behavioral data, to guide match of the actual behavior executed by the subject under analysis with the standard behavior executed under the behavioral situation.

15. The analysis and prompting method according to claim 13, further including prompting the standard behavior which has to be executed under the behavioral situation in accordance with the standard behavioral data when the actual behavior executed by the subject under analysis is abnormal, to guide match of the actual behavior executed by the subject under analysis with the standard behavior executed under the behavioral situation.

16. The analysis and prompting method according to claim 13, wherein the behavioral situation refers to an interaction situation between the subject under analysis and an interaction subject; the actual behavior refers to the actual behavior executed by the subject under analysis for the interaction subject; the standard behavior refers to the standard behavior executed by the subject under analysis for the interaction subject.

17. The analysis and prompting method according to claim 13, further including providing the acquisition module to acquire and use the actual behavior executed by the interaction subject to define the behavioral situation in which the subject under analysis is situated.

18. The analysis and prompting method according to claim 13, wherein, when the analysis module analyzes that the actual behavior executed by the subject under analysis is abnormal, the database is caused to store the actual situational data and the actual behavioral data for the prompt module to prompt the analysis result of the analysis module subsequently.

19. The analysis and prompting method according to claim 13, wherein, when the actual behavior executed by the subject under analysis is normal, stores the corresponding actual situational data and actual behavioral data in the database as the corresponding standard situational data and standard behavioral data stored in the database.

20. The analysis and prompting method according to claim 13, wherein, the analysis module searches for the standard situational data which match with the actual situational data from the database to predict a behavior which is going to be executed by the subject under analysis.
